(19) United States
(12) Patent Application Publication
(10) Pub. No.: US 2010/0256629 A1
(43) Pub. Date: Oct. 7, 2010

(54) METHODS AND DEVICES FOR TREATMENT OF THE OSTIUM

(75) Inventors: Bryan WYLIE, San Jose, CA (US); Edmund TAM, Mountain View, CA (US); Gregory SCHMITZ, Los Gatos, CA (US); Isidro M. GANDIONCO, Fremont, CA (US); Lee James CARMACK, Castro Valley, CA (US); Vahid SAADAT, Atherton, CA (US); Veerappan SWAMINATHAN, Sunnyvale, CA (US); Zachary J. MALCHANO, San Francisco, CA (US); Zachary WEST, Sunnyvale, CA (US)

Correspondence Address: LEVINE BAGADE HAN LLP, 2400 GENG ROAD, SUITE 120, PALO ALTO, CA, 94303, US

(73) Assignee: Voyage Medical, Inc., Redwood City, CA (US)

(21) Appl. No.: 12/755,244

(22) Filed: Apr. 6, 2010

Related U.S. Application Data
(60) Provisional application No. 61/167,016, filed on Apr. 6, 2009.

(51) Int. Cl. A61B 18/18

(52) U.S. Cl. 606/41

(57) ABSTRACT

Methods and devices for treatment of the ostium are described herein. Examples of such devices include inflatable balloons which have one or more raised pores along a distal portion which act as conduits for providing saline flow from the balloon to facilitate visualization of the contacted tissue as well as providing for a conduction path for energy delivery. The balloon may be configured in various shapes to facilitate contact of the balloon in and against the ostium.

### CROSS-REFERENCE TO RELATED APPLICATIONS

This application claims the benefit of priority to U.S. Prov. Pat. App. 61/167,016 filed Apr. 6, 2009, which is incorporated herein by reference in its entirety.

### FIELD OF THE INVENTION

The present invention relates generally to medical devices used for visualizing and/or assessing regions of tissue within a body. More particularly, the present invention relates to methods and apparatus for visualizing and/or assessing regions of tissue within a body, such as the chambers of a heart, to facilitate diagnoses and/or treatments for the tissue.

### BACKGROUND OF THE INVENTION

Conventional devices for accessing and visualizing interior regions of a body lumen are known. For example, ultrasound devices have been used to produce images from within a′body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

Moreover, many of the conventional imaging systems lack the capability to provide therapeutic treatments or are difficult to manipulate in providing effective therapies. For instance, the treatment in a patient's heart for atrial fibrillation is generally made difficult by a number of factors, such as visualization of the target tissue, access to the target tissue, and instrument articulation and management, amongst others.

Thus, such imaging balloons have many inherent disadvantages. For instance, such balloons typically lack mechanisms for treating a region of tissue visualized through the balloon. Treatment is often limited to ablation energy delivered by electrodes positioned along the balloon exterior or through laser energy transmitted directly through the balloon membrane. Contacting electrodes on the balloon surface against tissue, particularly tissue which may be moving such as a beating heart, may result in unsteady or uneven energy delivery while delivering laser energy often requires the balloon to remain in steady contact against the tissue region to be treated.

These types of imaging modalities are generally unable to provide desirable images useful for sufficient diagnosis and therapy of the endoluminal structure, due in part to factors such as dynamic forces generated by the natural movement of the heart. Accordingly, devices and methods which may effectively image underlying tissue while also effectively delivering energy to the tissue is desired.

### SUMMARY OF THE INVENTION

Generally, the ablation catheter assembly may comprise a catheter defining at least one lumen therethrough and an inflatable assembly positioned along the catheter. A guidewire may be advanced through catheter for guiding and positioning the assembly intravascularly and into position against the ostium of a vessel such as a pulmonary vein. The inflatable assembly may be inflated prior to placement against the ostium or after positioning the catheter in proximity to the ostium, if desired. The inflatable assembly may comprise an outer membrane having one or more openings, pores, or ports over a contact surface defined along a distal portion of the membrane. An inner membrane may be attached to the catheter while contained entirely within the outer membrane such that an annular space is formed between the outer and inner membranes. An outer membrane fluid port may also be defined along the catheter within the annular space to introduce a clear conductive fluid therethrough. Additionally, an imaging element such as an optical fiber or electronic imager (e.g., CCD, CMOS, etc.) may be positioned within the inner balloon or along the catheter such that the contacted and/or visualized tissue may be viewed through the clear fluid as well as through both inner and outer membranes. Either or both the outer and inner membranes may be fabricated from a clear and/or elastic material such as (but not limited to), e.g., polyurethane, silicone, etc.

As the fluid (e.g., a biocompatible liquid or inert gas such as saline or deuterium) is introduced within the inner membrane, one or more electrodes which are positioned along the inner membrane may be pushed out into proximity with the openings or pores of the outer membrane. A conductive fluid such as saline may be introduced into the annular space such that the conductive fluid may flow distally into the space and out the openings or pores. The imager within the balloon can be utilized to determine the appropriate level of inflation given that the tissue becomes clearly visible once the balloon is inflated such that it is in firm contact with the tissue. Upon the confirmation of adequate contact and a clear field of view, radiofrequency (RF) energy can be delivered via the electrodes and through the saline within the annular space to deliver ablative energy to the contacted underlying tissue.

Since the underlying tissue is ablated according to the flow of the saline, the raised openings or pores may provide specified pathways for the outflow of saline thereby controlling the development of lesions on the tissue surface of the ostium. Additionally, the space formed between the raised openings or pores may create channels for the blood flowing from the pulmonary vein to continue flowing throughout the procedure without completely occluding the blood flow.

Other variations of the balloon catheter may utilize one or more longitudinal or circumferential ridges which define one or more openings or pores therealong. The ridges may provide for improved re-direction of irrigation fluid through the openings or pores and into contact against the ostium as well as improved blood flow past the balloon between the ridges. Alternatively, one or more patches or groupings of openings or pores may be clustered around the distal portion of the balloon for contact against the ostium. Such a design may allow for expansion of the balloon in a manner to fit the anatomy securely.

In yet another variation, an electrode band may be defined circumferentially over a distal portion of the balloon where one or more openings or pores are circumferentially aligned along the band through the outer membrane. A rotatable fluid lumen may be optionally rotated within the balloon interior to direct a conduit opening adjacent to a selected opening along the electrode band to direct the irrigating conductive fluid to flow selectively from an individual opening.

Other variations may utilize a flexible frame which extends distally from the catheter along the interior of the balloon assembly. The frame may be comprised of a shape memory alloy which may be self expanding to allow for the frame and assembly to press fit securely against the ostium. Another variation may have one or more frame members extending radially with one or more corresponding wires attached at the free ends of each member. When tensioned, each of the members may be curved proximally, much like a crossbow, such that the assembly obtains a relatively lower profile for positioning within or against the ostium. The wire may be pushed or released such that the members may relax and extend radially relative to catheter such that the assembly obtains a larger profile and expands to conform to the shape of the ostium. Alternatively, pincer-like members may also be used.

Yet other variations may utilize one or more pivoting supports positioned within the assembly such that as the balloon is inflated into contact against the ostium each of the individual supports may pivot to conform to the underlying anatomy of the ostium.

In another alternative for securing the balloon against the ostium, the balloon itself may be modified in addition to or separate from the use of a frame. One example may include a balloon having one or more electrodes formed along a circumferential portion of the balloon which is recessed along a distal portion of the balloon. The openings along the recessed portion may each have a ring-shaped electrodes circumferentially positioned about the opening to provide the ablation energy.

Yet another variation of a balloon catheter may utilize an inner balloon which may be inflated at a pressure that is relatively higher than a pressure used to inflate the outer balloon. The outer balloon may be compliant enough to conform to the surface of the ostium while the relatively higher pressure inner balloon may be relatively stiffer to ensure that the balloon assembly is still readily positionable against the ostium without fear of buckling or collapsing the balloon assembly.

Yet another example includes an additional occluding balloon positioned along the catheter distal to the assembly. The occluding balloon may be placed within the vessel and inflated via a fluid or gas introduced through an opening along the catheter and into the balloon to serve as an anchor for the assembly. The occlusion balloon may also temporarily occlude the blood flow through the vessel. With blood flow temporarily occluded, the assembly may be inflated to position the openings against the ostium for ablation treatment. Optionally, the distal balloon and inflation assembly may be adjustable relative to one another via a telescoping section. Alternatively, an additional balloon may be positioned between the occlusion balloon and inflation assembly.

In yet another variation, the balloon may define one or more channels therethrough for shunting blood flow through the balloon thus eliminating the need to occlude the balloon and facilitating stabilization of the balloon relative to the ostium. Alternatively, the balloon may comprise a split chamber in which the balloon is inflated such that it both occludes the pulmonary vein and also engages and presses the one or more openings and electrodes directly onto the ostium.

In utilizing the imager for visualizing the underlying tissue, an optical fiber assembly or electronic imager (such as a CCD or CMOS imager) may be utilized. To facilitate the visualization of a relatively larger region of tissue, the imager may incorporate a convex lens positioned distal to the imager to create a fisheye lens effect that is able to visualize an increased viewing angle whilst affixed at a single spot inside the balloon.

### BRIEF DESCRIPTION OF THE DRAWINGS

FIGS. 1A and 1B show side and cross-sectional side views of one variation of a balloon catheter having one or more raised pores which act as conduits for providing a conductive fluid flow through which ablation energy may be conducted.

FIGS. 2 A and 2B show side and cross-sectional side views of another variation of a balloon catheter having one or more longitudinal ridges which define openings or pores through which a conductive fluid may flow.

FIG. 3 shows a side view of another variation of a balloon catheter having a circumferential ridge which defines openings or pores through which a conductive fluid may flow.

FIG. 4 shows a side view of another variation of a balloon catheter having one or more patches of openings or pores made of a relatively low durometer material through which a conductive fluid may flow.

FIG. 5 shows a side view of another variation of a balloon catheter having an electrode band around its distal end through which a conductive fluid may flow.

FIGS. 6A and 6B show side and perspective sectional views of another variation of a balloon catheter having one or more partitions within which segment saline flow into multiple sections.

FIG. 7 shows a perspective view of another variation of a balloon catheter having a rotatable fluid mechanism to direct saline flow to individual openings or pores.

FIG. 8 shows a perspective view of another variation of a balloon catheter having a flexible frame within the balloon and one or more openings or pores.

FIGS. 9A and 9B show cross-sectional side views of another variation of a balloon catheter having an expandable frame, like that of a crossbow, which may pivot internally at the distal end of the balloon.

FIG. 10 shows a cross-sectional side view of another variation of a balloon catheter having a pincer-like frame pivotable at its proximal end.

FIG. 11 shows a cross-sectional side view of another variation of a balloon catheter having one or more pivoting mechanisms supported along a frame which allow the balloon to conform to, e.g., the ostium of a pulmonary vein.

FIG. 12 shows a side view of another variation of a conically-shaped balloon catheter having a flexible frame within and one or more pores or openings.

FIG. 13 shows a side view of another variation of a balloon catheter having one or more openings or pores located near or at its distal end with a ring electrode surrounding each opening or pore.

FIG. 14 shows a cross-sectional side view of another variation of a balloon catheter having one or more openings or pores and an articulatable imaging element positionable in proximity to the openings or pores.

FIG. 15 shows a perspective view of another balloon variation having a plurality of raised openings or pores defined along a distal end.

FIGS. 16 A and 16B show cross-sectional side and end views of another variation of a double balloon catheter which maintains inflated balloons at two different pressures.

FIG. 17 shows a cross-sectional side view of another variation of a balloon catheter having a separate distal balloon.

FIG. 18 shows a cross-sectional side view of another variation of a balloon catheter having a telescoping distal balloon.

FIG. 19 shows a cross-sectional side view of another variation of a balloon catheter having electrodes positioned between the proximal and distal balloons.

FIG. 20 shows a cross-sectional side view of another variation of a balloon catheter having three balloon positioned along a catheter.

FIG. 21 shows a side view of another variation of a balloon catheter having one or more internal channels for redirecting blood flow.

FIG. 22 shows a side view of another variation of a balloon catheter having one or more pores for directing a conductive fluid.

FIG. 23 shows a side view of an imager (optical fiber or electronic) having a convex lens for increasing a viewing angle.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

The tissue-imaging and manipulation apparatus of the invention is able to provide real-time images in vivo of tissue regions within a body lumen such as a heart, which are filled with blood flowing dynamically through the region. The apparatus is also able to provide intravascular tools and instruments for performing various procedures upon the imaged tissue regions. Such an apparatus may be utilized for many procedures, e.g., visualizing and/or treating the ostium of vessels such as the ostia of the pulmonary veins for treating conditions such as atrial fibrillation. Disclosure and information regarding tissue visualization catheters generally which can be applied to the invention are shown and described in further detail in commonly owned U.S. patent application Ser. No. 11/259,498 filed Oct. 25, 2005 (U.S. Pat. Pub. 2006/0184048 A1), which is incorporated herein by reference in its entirety.

Aside from visualization and/or treatment of the ostium of a vessel, other procedures may be accomplished. Additional examples of such procedures are described in further detail in U.S. patent application Ser. No. 11/763,399 filed Jun. 14, 2007 (U.S. Pat. Pub. 2007/0293724 A1), which is incorporated herein by reference in its entirety. Additionally, details of tissue visualization and manipulation catheter which may be utilized with apparatus and methods described herein are described in U.S. patent application Ser. No. 11/259,498 filed Oct. 25, 2005 (U.S. Pat. Pub. 2006/0184048 A1), which is incorporated herein by reference in its entirety. Additional details and examples are further described in U.S. patent application Ser. No. 11/775,837 filed Jul. 10, 2007 (U.S. Pat. Pub. 2008/0009747 A1); Ser. No. 11/828,267 filed Jul. 25, 2007 (U.S. Pat. Pub. No. 2008/0033290 A1); Ser. No. 12/118,439 filed May 9, 2008 (U.S. Pat. Pub. 2009/0030412 A1); Ser. No. 12/201,811 filed Aug. 29, 2008 (U.S. Pat. Pub. 2009/0062790 A1); Ser. No. 12/209,057 filed Sep. 11, 2008 (U.S. Pat. Pub. 20090076498 A1); and Ser. No. 12/323,281 filed Nov. 25, 2008 (U.S. Pat. Pub. No. 2009/0143640 A 1), each of which may be utilized herewith. Each of these applications is incorporated herein by reference in its entirety.

In particular, such assemblies, apparatus, and methods may be utilized for treatment of various conditions, e.g., arrhythmias, through ablation under direct visualization. Details of examples for the treatment of arrhythmias under direct visualization which may be utilized with apparatus and methods described herein are described, for example, in U.S. patent application Ser. No. 11/775,819 filed Jul. 10, 2007 (U.S. Pat. Pub. No. 2008/0015569 A1), which is incorporated herein by reference in its entirety. Variations of the tissue imaging and manipulation apparatus may be configured to facilitate the application of bipolar energy delivery, such as radio-frequency (RF) ablation, to an underlying target tissue for treatment in a controlled manner while directly visualizing the tissue during the bipolar ablation process as well as confirming (visually and otherwise), appropriate treatment thereafter.

In utilizing an inflatable balloon for treatment of an ostium of a vessel, the inflatable balloon may generally comprise a visual electrode assembly which utilizes an expandable membrane which is enclosed except for one or more side purging ports through which the purging fluid may escape. One or more electrodes may be positioned along a support member or directly upon the balloon to deliver ablation energy conducted through the purging fluid and into or against the underlying tissue region to be treated in close proximity to the purging ports. Because the surface of the balloon membrane may be tapered, the assembly may be particularly suited for positioning against the ostium of a pulmonary vein so that the ablation energy discharged from the electrode may be directed through the purging fluid escaping through the one or more purging ports and into the tissue surrounding the ostium. A distal balloon anchor may be positioned along a distal tip or portion of the support catheter for advancement into the pulmonary vein to provide temporary anchoring for the assembly.

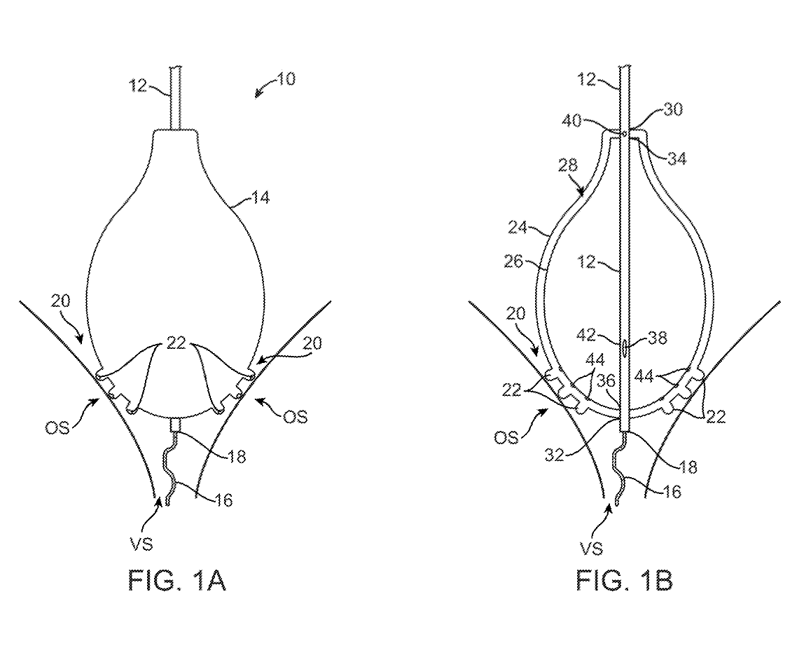

Generally, as shown in the side and cross-sectional side views of FIGS. 1A and 1B, the ablation catheter assembly 10 may comprise a catheter 12 defining at least one lumen 18 therethrough and an inflatable assembly 14 positioned along the catheter 12. A guidewire 16 may be advanced through catheter 12 for guiding and positioning the assembly 14 intravascularly and into position against the ostium OS of a vessel VS such as a pulmonary vein. The inflatable assembly 14 may be inflated prior to placement against the ostium OS or after positioning catheter 12 in proximity to the ostium OS, if desired.

The inflatable assembly 14 may comprise an outer membrane 24 attached at its proximal end 30 and distal end 32 to the catheter 12 and the outer member 24 may also define one or more openings, pores, or ports 22 over a contact surface 20 defined along a distal portion of the membrane 24. These openings or pores 22 may each comprise a raised portion over a circumference of the membrane 24. An inner membrane 26 may be attached to the catheter 12 along its proximal 34 and distal end 36 while contained entirely within outer membrane 24 such that an annular space 28 is formed between the outer 24 and inner membranes 26. The catheter 12 may define an inner membrane fluid port 38 through which a clear fluid may be introduced into the interior of the inner membrane 26 to inflate or expand the inner balloon. An outer membrane fluid port 40 may also be defined along the catheter 12 within the annular space 28 to introduce a clear conductive fluid therethrough. Additionally, an imaging element 42 such as an optical fiber or electronic imager (e.g., CCD, CMOS, etc.) may be positioned within the inner balloon or along the catheter 12 such that the contacted and/or visualized tissue may be viewed through the clear fluid as well as through both inner 26 and outer 24 membranes. Either or both the outer 24 and inner 26 membranes may be fabricated from a clear and/or elastic material such as (but not limited to), e.g., polyurethane, silicone, etc.

As the fluid (e.g., a biocompatible liquid or inert gas such as saline or deuterium) is introduced within inner membrane 26 through opening 38, one or more electrodes 44 which are positioned along the inner membrane 26 may be pushed out into proximity with the openings or pores 22 of the outer membrane 24. A conductive fluid such as saline may be introduced through opening 40 into the annular space 28 such that the conductive fluid may flow distally into the space and out the openings or pores 22. The imager 42 within the balloon can be utilized to determine the appropriate level of inflation given that the tissue becomes clearly visible once the balloon is inflated such that it is in firm contact with the tissue. Upon the confirmation of adequate contact and a clear field of view, radiofrequency (RF) energy can be delivered via the electrodes 44 and through the saline within the annular space 28 to deliver ablative energy to the contacted underlying tissue. Further examples for delivering ablation energy conducted through a fluid are described in further detail in Ser. No. 12/201,811 filed Aug. 29, 2008 (U.S. Pat. Pub. 2009/0062790 A1), which has been incorporated herein by reference.

Since the underlying tissue is ablated according to the flow of the saline, the raised openings or pores 22 may provide specified pathways for the outflow of saline thereby controlling the development of lesions on the tissue surface of the ostium OS. Additionally, the space formed between the raised openings or pores 22 may create channels for the blood flowing from the pulmonary vein to continue flowing throughout the procedure without completely occluding the blood flow.

In treating conditions such as atrial fibrillation, studies have shown that it is generally advantageous to create a conduction block around the ostium OS of the pulmonary vein in the left atrium of the heart. A conduction block may be created by a variety of methods that include but are not limited to direct application of not only RF energy, but also laser energy, ultrasound energy, cryo-ablative energy, etc.

With the introduction of an irrigating fluid, hematocrit and the chances of clotting may be potentially reduced during such a procedure. Additionally, because the inflation fluid within inner membrane 26 and the irrigating fluid within the annular space 28 are clear, visualization of the tissue area may be maintained during the procedure and further allows for the unobstructed and uniform delivery of ablative energy. Moreover, the irrigating fluid may also cool the surface of the ostium OS potentially preventing overheating or burning of the tissue or coagulation.

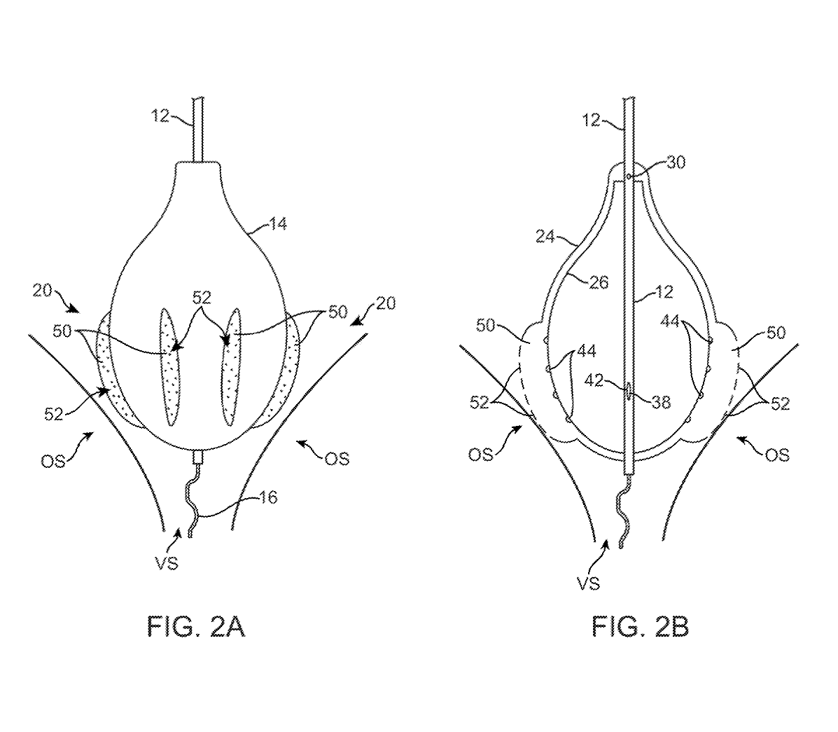

FIGS. 2A and 2B show side and cross-sectional side views of another variation of a balloon catheter where the outer membrane 24 of inflation assembly 14 may have one or more longitudinal ridges 50 which extend along the contact surface 20 for contact against the ostium OS. Each of the ridges 50 may define one or more openings or pores 52 therealong where the ridges may be pre-fabricated or made of a relatively lower durometer clear elastic material which compresses and complies with the ostium OS. The ridges 50 may provide for improved re-direction of irrigation fluid through the openings or pores 52 and into contact against the ostium OS as well as improved blood flow past the balloon between the ridges 50.

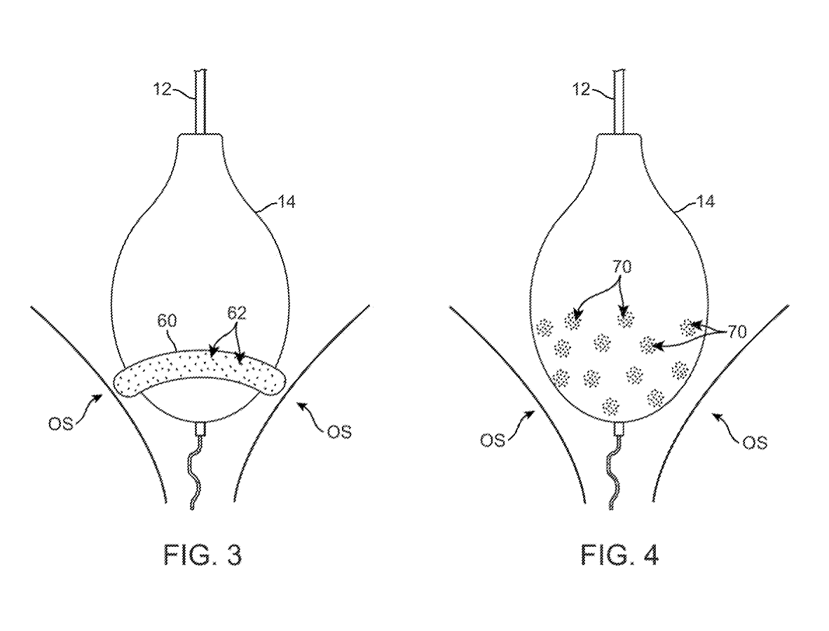

FIG. 3 shows yet another variation in which the ridge 60 may be formed circumferentially over the contact region with the openings or pores 62 defined along the circumferential ridge 60. Ridge 60 may extend partially over the balloon or it may extend entirely around the balloon circumference. This ridge 60 may provide for the formation of a circumferential lesion can be formed upon the ostium OS in a single treatment.

FIG. 4 shows a side view of another variation where one or more patches or groupings of openings or pores 70 may be clustered around the distal portion of the balloon for contact against the ostium OS. A single durometer outer balloon may be interspersed with patches of a relatively lower durometer material over the distal contact portion. These patches may be relatively more elastic and compliant such that when the irrigation fluid is introduced into the outer balloon, the lower durometer pores 70 may expand to take the shape of the contour of the PV ostium and allow saline to pass through for ablating the underlying ostium OS. Such a design may allow for expansion of the balloon in a manner to fit the anatomy securely.

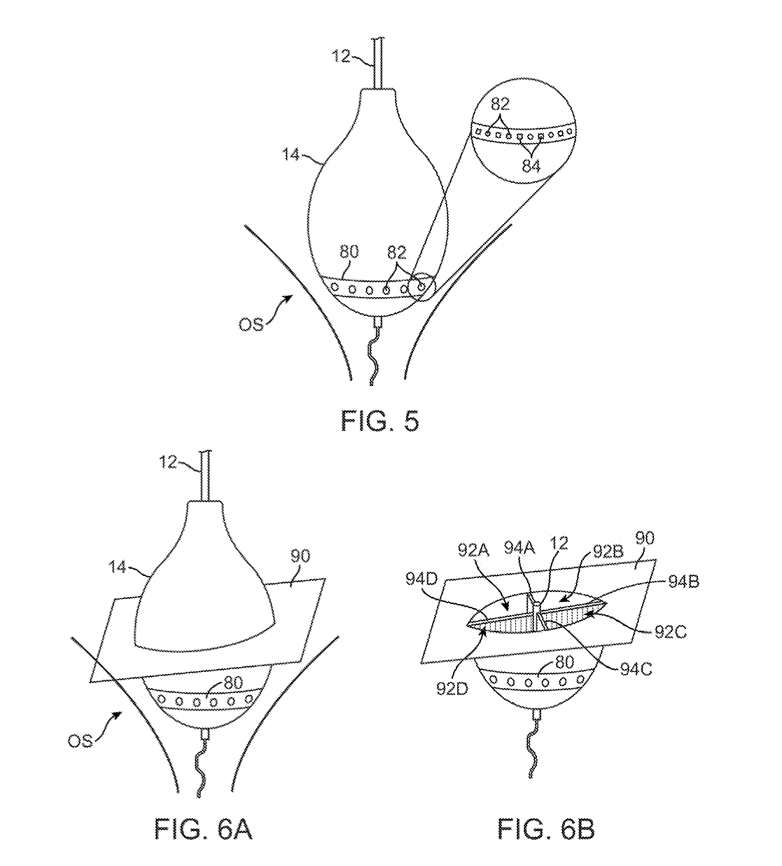

FIG. 5 shows a side view and a detail view of another variation of the balloon catheter having an electrode band 80 defined circumferentially over a distal portion of the balloon. Electrode band 80 may define one or more openings or pores 82 which are circumferentially aligned along the band 80 through the outer membrane. One or more electrodes 84 may be positioned adjacent to the corresponding openings or pores 82 in an alternating manner such that as the irrigating fluid is passed through the balloon assembly 14 and out through the openings 82, the energy from the adjacent electrodes 84 may be conducted through the fluid and into the contacted ostium OS.

FIGS. 6A and 6B show side and perspective sectional views of another variation of a balloon catheter having an electrode band 80 located about a distal portion of the balloon and an interior which is partitioned to channel irrigating fluid flow into segments. As shown along the plane of separation 90 in FIG. 6B, one or more partitions 94A, 94B, 94C, 94D may extend from the catheter 12 to the balloon interior surface to separate the interior into two or more corresponding chambers 92A, 92B, 92C, 92D. Partitioning the balloon interior may allow for the fluid to be selectively flowed either through the partitions or through the chambers to selectively conduct the energy through one or more selected region of the balloon.

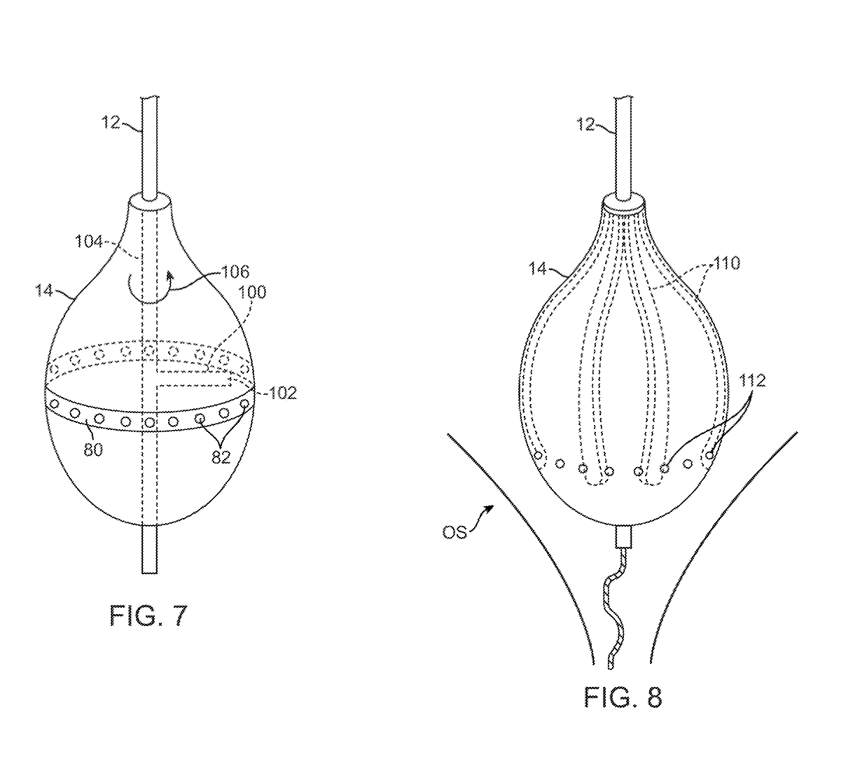

FIG. 7 shows a perspective view of another balloon catheter variation where catheter 12 may comprise a rotatable fluid lumen 104 which may be rotated within the balloon interior, e.g., in a direction of rotation 106, to direct a conduit opening 102 of a fluid conduit 100 which extends from the fluid lumen 104. Fluid conduit 100 may be selectively rotated to position conduit opening 102 adjacent to a selected opening 82 along electrode band 80 to direct the irrigating conductive fluid to flow selectively from an individual opening. This fluid flow out of a particular opening may allow for the conduction of energy through selected individual openings to treated particular region of tissue along the ostium OS.

When performing visualization and treatment with a balloon inside a heart, the tissue surface is constantly moving in accordance with the heart beat and there is an outflow of blood from the pulmonary vein. Moreover, the ostium of one or more of the pulmonary veins may be irregularly shaped in an inconsistent manner between patients. These factors, amongst others, typically contribute to the dislodgement of balloons from the ostium. Thus, mechanisms may be utilized for maintaining contact between the balloon and ostium surface.

FIG. 8 shows one example of a flexible frame 110 which may extend distally from catheter 12 along the interior of the balloon assembly 14. The frame 110 may be comprised of a shape memory alloy such as Nickel-Titanium (Nitinol) or a flexible plastic, etc., which may be self expanding to allow for the frame 110 and assembly 14 to press fit securely against the ostium OS. The frame 110 may extend distally along the entire length or a partial length of the assembly 14 such that when fully expanded, the openings 112 along balloon 14 may be pressed into contact or in proximity to the ostium OS in a secure manner.

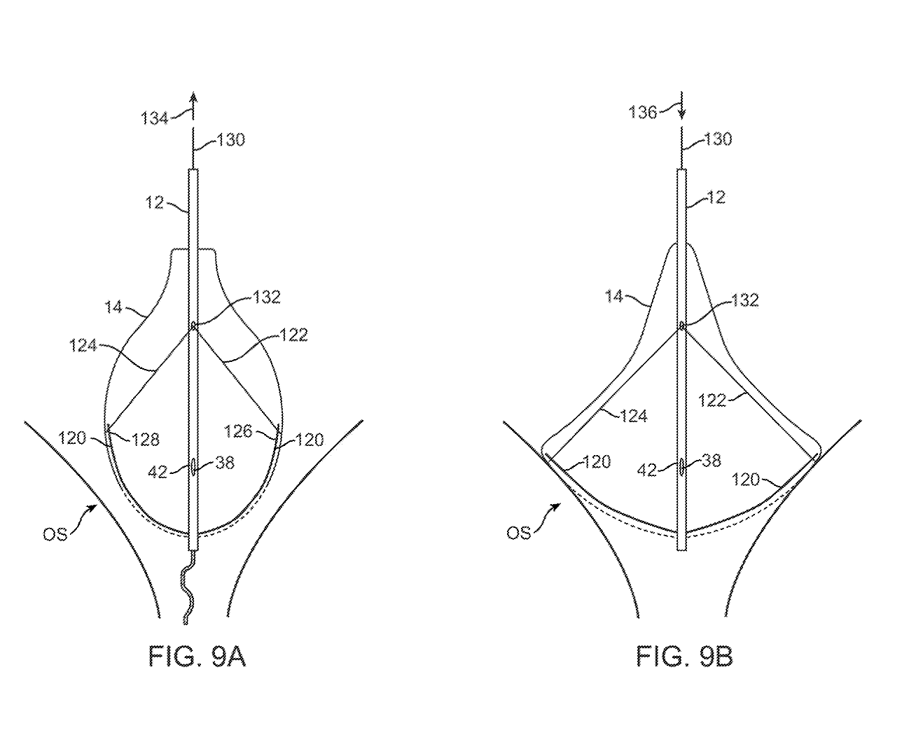

FIGS. 9A and 9B show cross-sectional side views of another variation where a frame mechanism having one or more flexible frame members 120 may be attached to the catheter 12 near or at a distal end of the assembly 14 within or along the balloon. Each of the frame members 120 may extend radially with one or more corresponding wires 122, 124 attached at the free ends of each member 120 along attachment points 126, 128 such that each wire passes through an opening 132 defined along the catheter 12 within the balloon interior and to one or more push/pull wires 130 extending proximally through the catheter 12. When wire 130 is tensioned, as illustrated by the direction of tension 134, each of the members 120 may be curved proximally, much like a crossbow, such that the assembly 14 obtains a relatively lower profile for positioning within or against the ostium OS. The wire 130 may be pushed or released, as indicated by the direction of release or compression 136, such that the members 120 may relax and extend radially relative to catheter 12 such that the assembly 14 obtains a larger profile and expands to conform to the shape of the ostium OS, as shown in FIG. 9B. The underlying tissue may then be visualized, e.g., via imager 42, and treated accordingly as described herein with the conductive irrigation fluid.

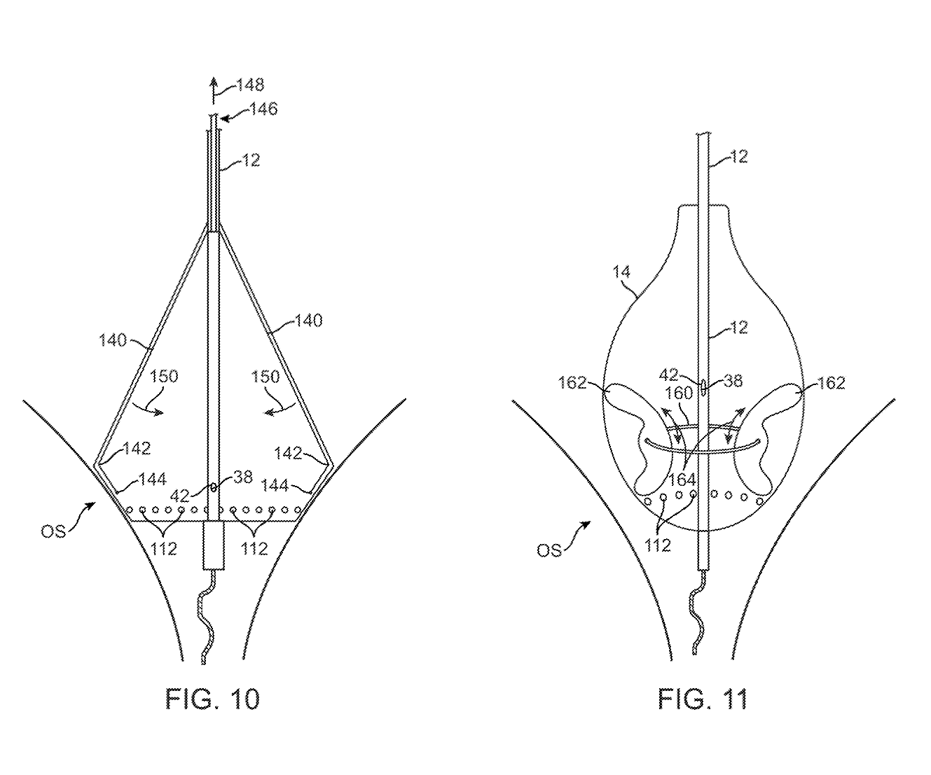

FIG. 10 shows another variation having one or more frame members 140 within or along the balloon where each member 140 may have an angle 142 such that the frame is shaped in a pincer-like configuration. One or more wires 146 positioned through the catheter 12 may be attached to the frame members 140 such that pulling the wires 146, e.g., in a direction of tension 148, may collapse or urge the pincer closed, as indicated by the direction of collapse 150, to collapse the balloon upon itself. In use, during delivery, the wires 146 may be pulled proximally and the pincer is closed but once the ostium OS is reached, the wire 146 may be released or pushed to then expand the frame members 140 and the balloon to press securely against the ostium OS. The level of control to which the frame may be opened may be provided by the operator in contorting the shape of the balloon such that undue force is avoid when positioning against the ostium OS. Additionally, electrodes 144 may be placed near or at the distal ends of the frame members 140 to provide the energy through the irrigation fluid passed through the openings 112.

FIG. 11 shows another variation of a balloon catheter having one or more pivoting supports 162, which may be crescent-shaped or arcuate in configuration, each rotatably coupled along a circumferential support member 160. The pivoting supports 162 may be positioned within the assembly 14 such that as the balloon is inflated into contact against the ostium OS each of the individual supports 162 may pivot, as indicated by the direction of rotation 164, to conform to the underlying anatomy of the ostium OS. The shape and design of the pivoting supports 162 enable the balloon to fit securely against the ostium OS and the conformed shape may be held in place so long as sufficient force is applied on the balloon in the direction towards the tissue surface.

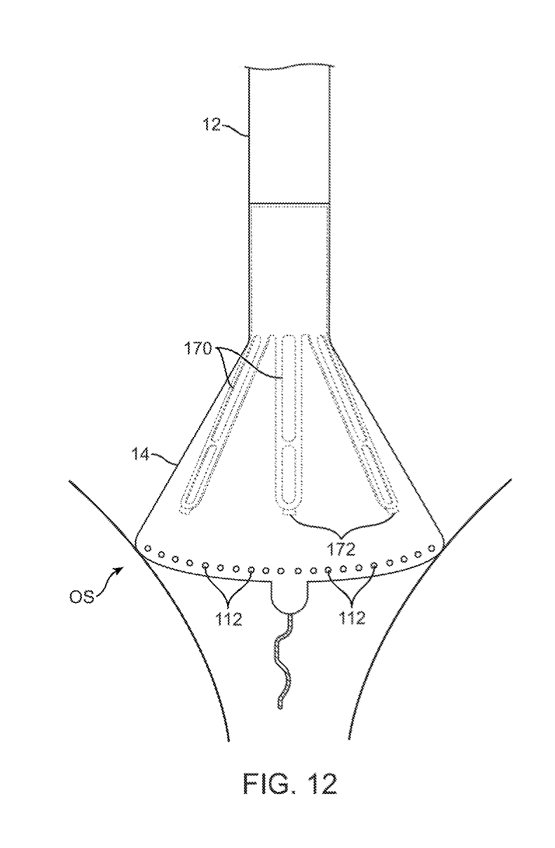

FIG. 12 shows a side view of another variation where the assembly 14 may be shaped in a conical configuration for expansion against the ostium OS. The openings 112 may be positioned circumferentially along a distal portion of the assembly 14 which contacts the tissue. A support frame 170 made of a flexible material such as Nitinol may have one or more electrodes 172 positioned along the frame 170 for delivering the energy through the irrigation fluid. The configuration of assembly 14 is shown and described in further detail in, e.g., U.S. patent application Ser. No. 11/687,597 filed Mar. 16, 2007 (U.S. Pat. App. 2007/0287886 A1) or Ser. No. 11/259,498 filed Oct. 25, 2005 (U.S. Pat. App. 2006/0184048 A1), which are incorporated herein by reference in its entirety.

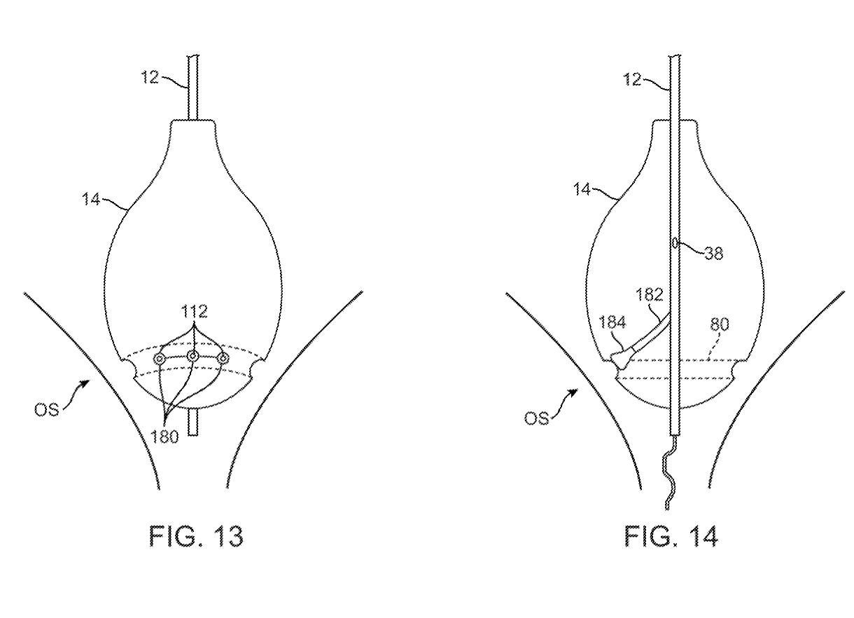

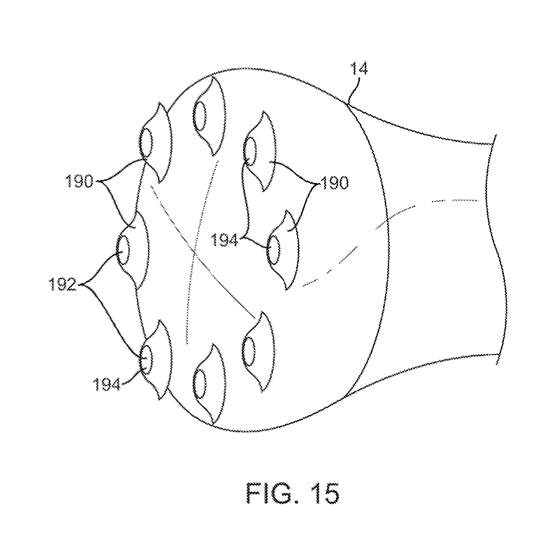

In another alternative for securing the balloon against the ostium, the balloon itself may be modified in addition to or separate from the use of a frame. One example is shown in the side view of FIG. 13 which shows a balloon having one or more electrodes formed along a circumferential portion of the balloon which is recessed along a distal portion of the balloon. The openings 112 along the recessed portion may each have a ring-shaped electrodes 180 circumferentially positioned about the opening 112 to provide the ablation energy. As the balloon is pressed against the ostium OS, fluid may still flow through the openings and around the recessed portion for transmitting the energy in a circumferential pattern. FIG. 14 shows another variation which also defines a recessed portion along the balloon. An articulatable support member 182 may be selectively positioned along the interior of the assembly to position an imaging element 184 in proximity to the recessed portion for visualizing the treated tissue.

FIG. 15 shows another of a balloon assembly 14 having a conical shape and a plurality of projections 190 with openings 192 defined each projection 190 over a distal surface of the assembly 14. A corresponding electrode 194 may also be positioned in proximity to the openings 192 for delivering the ablation energy through the irrigation fluid.

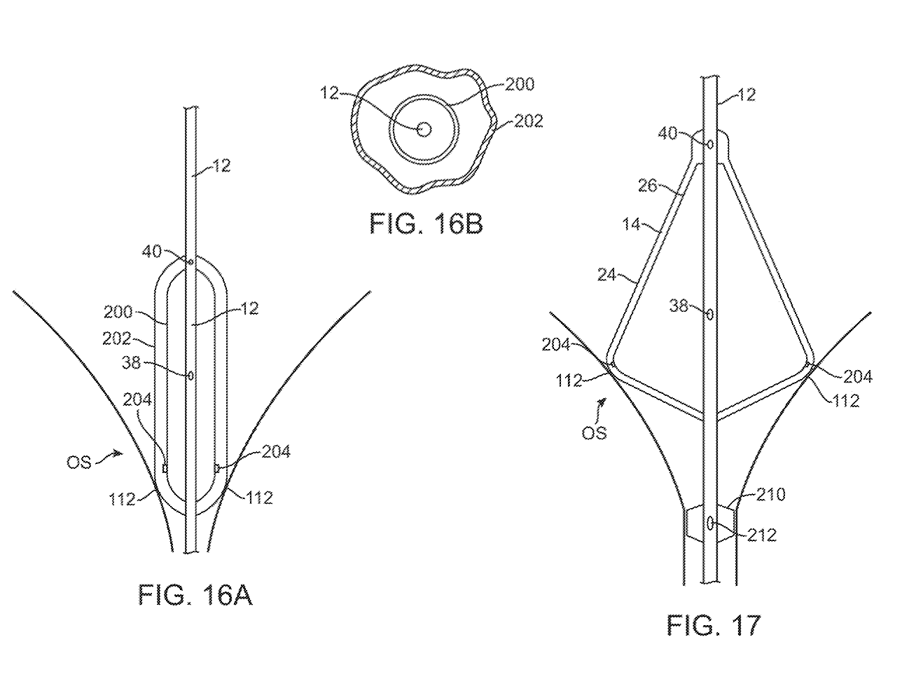

FIGS. 16A and 16B show cross-sectional side and end views, respectively, of yet another variation of a balloon catheter which may utilize an inner balloon 200 which may be inflated at a pressure that is relatively higher than a pressure used to inflate the outer balloon 202. The outer balloon 202 may be compliant enough to conform to the surface of the ostium OS while the relatively higher pressure inner balloon 200 may be relatively stiffer to ensure that the balloon assembly is still readily positionable against the ostium OS without fear of buckling or collapsing the balloon assembly. One or more electrodes 204 may be positioned along the inner balloon 200 to provide the energy for the irrigation fluid introduced through the outer balloon 202 and through the one or more openings 112.

Yet another example is shown in the cross-sectional view of FIG. 17 which shows a balloon assembly 14 having an additional occluding balloon 210 positioned along the catheter 12 distal to the assembly 14. The occluding balloon 210 may be placed within the vessel and inflated via a fluid or gas introduced through an opening 212 along the catheter 12 and into balloon 210 to serve as an anchor for the assembly 14. Occlusion balloon 210 may also temporarily occlude the blood flow through the vessel. With blood flow temporarily occluded, the assembly 14 may be inflated to position the openings 112 against the ostium OS for ablation treatment.

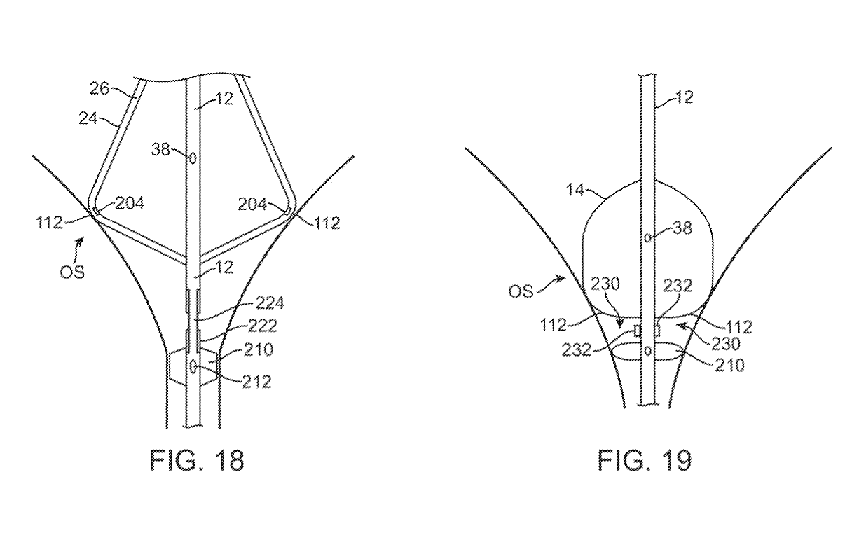

FIG. 18 shows a cross-sectional side view of another variation similar to the variation of FIG. 17 but with the addition of a telescoping section 220 defined along the catheter 12 between inflation assembly 14 and occlusion balloon 210. The telescoping section 220 may have a central segment 224 upon which a translating segment 222 may slide longitudinally to allow for adjustment of a position of inflation assembly 14 relative to occlusion balloon 210.

FIG. 19 shows yet another variation having a compliant occlusion balloon 210 which is extremely pliable. The occlusion balloon 210 may be first inflated within the vessel and the inflation assembly 14 may then be inflated while the irrigation fluid is also introduced through the openings 112 into the isolated space 230 formed between the assembly 14 and occlusion balloon 210. The conductive irrigation fluid may displace the blood within the isolated space 230 such that one or more electrodes 232 positioned along the catheter within the isolated space 230 may then be used to deliver energy through the irrigation fluid and to the tissue defined between the balloons along the isolated space 230.

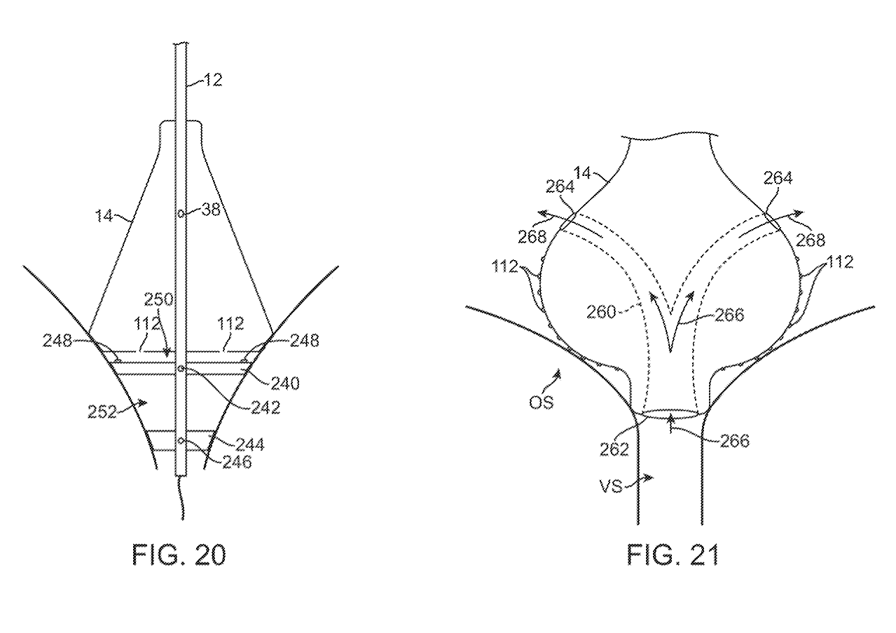

FIG. 20 shows a cross-sectional side view of yet another variation of a balloon catheter having an inflation assembly 14, a distal occlusion balloon 244 (inflatable via opening 246 defined along catheter 12), and an additional middle balloon 240 (inflatable via opening 242 defined along catheter 12). The use of three balloons creates not only a distal isolated space 252 but also a proximal isolated space 250 each of which may be purged of blood with the irrigation fluid. The presence of one or more electrodes 248 in either or both of the spaces 250, 252 may be used to deliver ablation energy through the irrigation fluid and into the underlying tissue regions of the ostium OS. Additionally, the middle balloon 240 may also be configured as a porous or “weeping” balloon which may limit the need for the irrigation fluid to be used to create the fluid environment and may also allow for a relatively thinner lesion to be created when RF energy is transmitted from the electrodes through the fluid filled environment to the tissue surface.

FIG. 21 shows yet another variation of an inflation assembly 14 which may define one or more channels through the balloon for shunting blood flow through the balloon thus eliminating the need to occlude the balloon and facilitating stabilization of the balloon relative to the ostium OS. In this example, an opening 262 may be defined at a distal end of the balloon such that a channel 260 may be defined through the balloon. Channel 260 may become divided into two or more channels with respective openings 264 defined along a proximal end of the balloon to allow for the entering blood flow 266 from the pulmonary vein to be redirected 268 into the left atrial chamber of the heart.

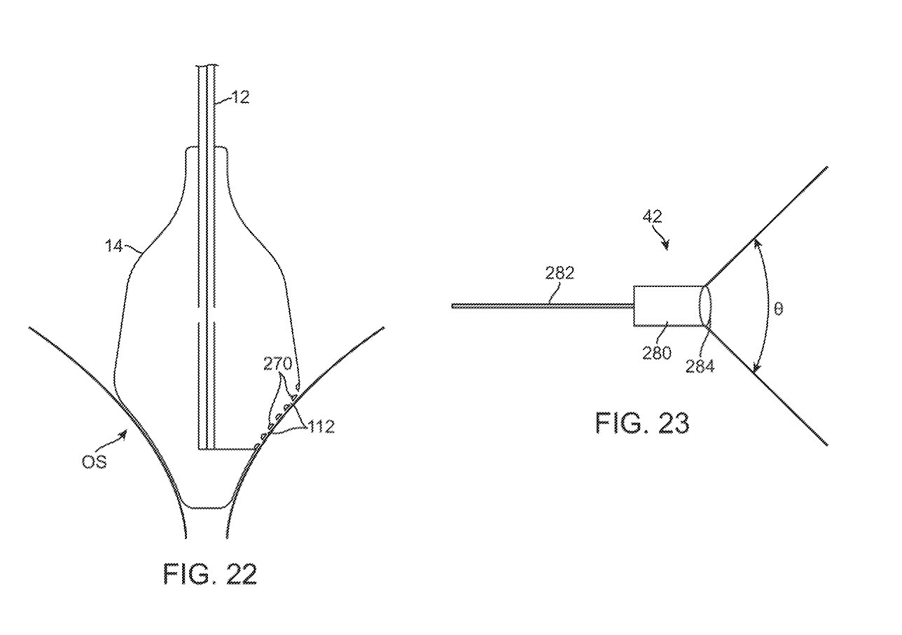

FIG. 22 shows a cross-sectional side view of yet another balloon catheter variation which may maintain contact against the ostium OS by a split chamber balloon in which the balloon is inflated such that it both occludes the PV and also engages and presses the one or more openings 112 and electrodes 270 directly onto the ostium OS.

In utilizing the imager for visualizing the underlying tissue, an optical fiber assembly or electronic imager (such as a CCD or CMOS imager) may be utilized. To facilitate the visualization of a relatively larger region of tissue, an imager 280 such as the variation shown in the side view of FIG. 23 may be used in any of the balloon assemblies described herein. Imager 280 may be an optical fiber assembly, in which case connection 282 may comprise a length of optical fibers. Alternatively, imager 280 may comprise an electronic imager, in which case connection 282 may comprise a conductor. In either case, imager 280 may comprise a convex lens 284 positioned distal to the imager 280 to create a fisheye lens effect that is able to visualize an increased viewing angle θ whilst affixed at a single spot inside the balloon.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue treatment device configured to treat an ostium of a vessel, comprising:
an elongate catheter;
an expandable inner membrane attached along a distal end of the catheter and in fluid communication with a first opening along the catheter;
an expandable outer membrane attached along the distal end such that the inner membrane is enclosed by the outer membrane and is in fluid communication with a second opening along the catheter, wherein the outer membrane and inner membrane form an annular space therebetween which is in fluid communication with an environment external to the outer membrane through one or more openings defined along a distal portion of the outer membrane; and,
one or more electrodes in proximity to the one or more openings such that the electrodes are in communication with the environment.

2. The device of claim 1 further comprising an imager positioned within or along the catheter for visualizing the ostium when contacted against the outer membrane.

3. The device of claim 1 wherein the expandable inner membrane comprises an inflatable balloon.

4. The device of claim 1 wherein the expandable outer membrane defines one or more raised ports.

5. The device of claim 1 wherein the one or more electrodes are positioned along an exterior of the inner membrane.

6. The device of claim 1 wherein the one or more electrodes are positioned about the one or more openings.

7. The device of claim 1 further comprising a conductive fluid which is introduced through the second opening and through the annular space such that the fluid contacts the one or more electrodes.

8. The device of claim 1 wherein the outer membrane defines one or more ridges which protrude from an external surface of the outer membrane such that the one or more openings are defined along the ridges.

9. The device of claim 8 wherein the one or more ridges are defined longitudinally or circumferentially along the external surface relative to the device.

10. The device of claim 1 wherein the one or more openings are clustered within one or more defined patches along the outer membrane.

11. The device of claim 1 wherein the one or more openings are defined circumferentially about the outer membrane.

12. The device of claim 1 further comprising one or more partitions within the inner membrane.

13. The device of claim 1 further comprising a rotatable member within the inner membrane which is selectively positionable into proximity of the one or more openings.

14. The device of claim 1 further comprising a flexible frame within or along the device such that the frame is movable between a low-profile configuration and a deployed configuration.

15. The device of claim 1 further comprising an inflatable occlusion balloon attached to the catheter distal to the outer membrane.

16. The device of claim 15 wherein a portion of the catheter is adjustable in length between the occlusion balloon and outer membrane.

17. The device of claim 1 wherein the device defines one or more channels from a distal end of the device to a proximal end of the device.

18. A method of treating an ostium of a vessel, comprising:
expanding an inner membrane and an outer membrane enclosing the inner membrane each attached along a distal end of the catheter within or against an ostium of a vessel;
introducing a conductive fluid into an annular space defined between the inner membrane and the outer membrane such that the fluid passes through one or more openings defined along the outer membrane and into an environment external thereto;
actuating one or more electrodes positioned in proximity to the one or more openings such that energy is delivered through the fluid and into the ostium external to the outer membrane; and,
visualizing the ostium through the outer membrane.

19. The method of claim 18 wherein expanding an inner membrane comprises inflating the inner membrane via a transparent gas or fluid.

20. The method of claim 18 wherein expanding an inner membrane comprises expanding a flexible frame within or along the inner membrane.

21. The method of claim 18 wherein introducing a conductive fluid comprises introducing saline fluid into the annular space.

22. The method of claim 18 wherein introducing a conductive fluid comprises passing the fluid through one or more openings which are raised relative to a surface of the outer membrane.

23. The method of claim 18 wherein actuating one or more electrodes comprises actuating the one or more electrodes positioned along an exterior surface of the inner membrane and in proximity to the one or more openings.

24. The method of claim 18 wherein actuating one or more electrodes comprises actuating the one or more electrodes positioned about each of the one or more openings.

25. The method of claim 18 wherein visualizing the ostium comprises visualizing via an imager positioned along the catheter within the inner membrane.
